(19) United States
(12) Patent Application Publication
(10) Pub. No.: US 2009/0042898 A1
(43) Pub. Date: Feb. 12, 2009

(54) USE OF COMPOUNDS ACTIVE ON THE SIGMA RECEPTOR FOR THE TREATMENT OF MECANICAL ALLODYNIA

(75) Inventor: Jose Manuel Baeyens Cabrera, Granada (ES)

Correspondence Address: BANNER & WITCOFF, LTD., 1100 13th STREET, N.W., SUITE 1200, WASHINGTON, DC, 20005-4051, US

(73) Assignee: LABORATORIOS DEL DR. ESTEVE S.A., Barcelona (ES)

(21) Appl. No.: 11/658,153

(22) PCT Filed: Jul. 25, 2005
(86) PCT No.: PCT/EP2005/008080

§ 371 Date: Jun. 5, 2008

Related U.S. Application Data
(63) Continuation of application No. 10/902,273, filed on Jul. 30, 2004, now abandoned.
Application No. 10/902,273 is a continuation of application No. 10/902,272, filed on Jul. 30, 2004, now abandoned.

(30) Foreign Application Priority Data
Jul. 24, 2004 (EP) 04017561.4
Jul. 24, 2004 (EP) 04017562.2
Aug. 27, 2004 (EP) 04020376.2

(51) Int. Cl. A61K 31/495; A61K 31/135

(52) U.S. Cl. 514/252.12; 514/646

(57) ABSTRACT

The present invention refert to the use of compounds active on the sigma receptor for the treatment of mechanical allodynia.

### FIELD OF THE INVENTION

The present invention refers to the use of compounds active on the sigma receptor for the treatment of the symptoms of mechanical allodynia, as well as treatment of the disease causing the symptoms, the prevention or the prophylaxis of the symptoms of mechanic allodynia, as well as the prevention or the prophylaxis of the disease causing the symptoms.

### BACKGROUND OF THE INVENTION

The treatment of pain conditions is of great importance in medicine. There is currently a world-wide need for additional pain therapy. The pressing requirement for a specific treatment of pain conditions or as well a treatment of specific pain conditions which is right for the patient, which is to be understood as the successful and satisfactory treatment of pain for the patients, is documented in the large number of scientific works which have recently and over the years appeared in the field of applied analgesics or on basic research on nociception.

PAIN is defined by the International Association for the Study of Pain (IASP) as “an unpleasant sensory and emotional experience associated with actual or potential tissue damage, or described in terms of such damage (IASP, Classification of chronic pain, 2^{nd} Edition, IASP Press (2002), 210). Even though pain is always subjective its causes or syndromes can be classified.

Especially mechanical allodynia which in the past years has developed into a major health problem in broad areas of the population needs a very specific treatment, especially considering that any treatment of mechanical allodynia is extremely sensitive to the causes behind the pain, be it the disease ultimately causing it or the mechanistic pathway over which it develops.

Therefore, it was the underlying problem solved by this invention to find new ways of treating mechanical allodynia.

So, the main object of this invention is the use of a compound binding to the sigma receptor in the production of a medicament for the treatment of mechanical allodynia.

This/these compound/s may be in neutral form, the form of a base or acid, in the form of a salt, preferably a physiologically acceptable salt, in the form of a solvate or of a polymorph and/or in the form of in the form of its racemate, pure stereoisomers, especially enantiomers or diastereomers or in the form of mixtures of stereoisomers, especially enantiomers or diastereomers, in any suitable mixing ratio.

While working on compounds binding to the sigma receptor and with models like knock-out mice it was surprisingly found out that mechanical allodynia is connected to the sigma receptor so that compounds binding to the sigma receptor were acting on mechanical allodynia with a high potency.

“Treating” or “treatment” as used in this application are defined as including the treatment of the symptoms—of mechanical allodynia—as well as treatment of the disease or disease consequences causing the symptoms, the prevention or the prophylaxis of the symptoms—of mechanical allodynia—as well as the prevention or the prophylaxis of the disease or disease consequences causing the symptoms. Preferably “treating” or “treatment” as used in this application are defined as including the treatment of the symptoms—of mechanical allodynia—as well as treatment of the disease consequences causing the symptoms, the prevention or the prophylaxis of the symptoms—of mechanical allodynia—as well as the prevention or the prophylaxis of the disease consequences causing the symptoms. Most preferably “treating” or “treatment” as used in this application are defined as including the treatment of the symptoms of mechanical allodynia, and the prevention or the prophylaxis of the symptoms of mechanical allodynia.

“The sigma receptor/s” as used in this application is/are well known and defined using the following citation: This binding site represents a typical protein different from opioid, NMDA, dopaminergic, and other known neurotransmitter or hormone receptor families (G. Ronsisvalle et al. Pure Appl. Chem. 73, 1499-1509 (2001)). Pharmacological data based on ligand binding studies, anatomical distribution and biochemical features distinguish at least two subtypes of σ receptors (R. Quiron et al., Trends Pharmacol. Sci. 13, 85-86 (1992); M. L. Leitner, Eur. J. Pharmacol. 259, 65-69 (1994); S. B. Hellewell and W. D. Bowen; Brain Res. 527, 244-253 (1990)) (G. Ronsisvalle et al. Pure Appl. Chem. 73, 1499-1509 (2001)). The protein sequence of sigma receptors (Sigma 1 (σ1) and Sigma 2 (σ2)) is known (e.g. Prasad, P. D. et al., J. Neurochem. 70 (2), 443-451 (1998)) and they show a very high affinity for e.g. pentazocine.

“Compound/s binding to the sigma receptor” or “sigma ligand” as used in this application is/are defined as having an IC₅₀ value of ≦5000 nM, more preferably ≦1000 nM, more preferably ≦500 nM. More preferably, the IC₅₀ value is ≦250 nM. More preferably, the IC₅₀ value is ≦100 nM. Most preferably, the IC₅₀ value is ≦50 nM. Additionally, the wording “Compound/s binding to the sigma receptor”, as used in the present application is defined as having at least ≧b 50% displacement using 10 mM radioligand specific for the sigma receptor (e.g. preferably ¹H-pentazocine) whereby the sigma recepor may be any sigma receptor subtype. Preferably, said compounds bind to the sigma-1 receptor subtype.

Compounds binding to the sigma receptor generally also known as sigma ligands are well known in the art with many of them falling under the definition for “Compound/s binding to the sigma receptor” set up above. Still even though there are many uses known for sigma ligands such as antipsychotic drugs, anxiolytics, antidepressants, the treatment of stroke, antiepileptic drugs and many other indications including anti-migraine and general pain (mostly analgesia) there is nowhere any mentioning of these compounds being useful against mechanical allodynia.

Preferably, compounds selected from the group consisting of amitriptyline, desipramine, fluoxetine, methadone and tiagabine, are disclaimed from the present invention. These compounds have been shown to bind to the sigma receptor and have an IC50 value ≧100 nM.

Preferably, compounds selected from the group consisting of agmatine, alfentanil, all-trans retinoic acid (ATRA), Erythropoietin, Etanercept, GV196771, GV196771A, GW406381X, KRN5500, L-N (6)-(1-iminoethyl) lysine (L-NIL), LY379268, LY389795, neurotropin, N-methyl-D-aspartic acid, peptide analog of thymulin (PAT), Propentofylline, ReN-1869 [(R)-1-(3-(10,11-dihydro-5H-dibenzo[a,d]cyclohepten-5- ylidene)-1-propyl)-3-piperidine carboxylic acid], R-phenylisopropyl-adenosine (R-PIA), SD-282, Semaphorin3A, SHU9119, T62 (2-amino-3-(4-chlorobenzoyl)-5,6,7,8- tetrahydrobenzothiophene) and ziconotide are disclaimed from the present invention. The majority of these compounds has an IC₅₀≧100 nM with respect to binding the sigma receptor.

Compounds binding to the sigma receptor known in the art and matching the criteria of sigma ligand (i.e. having an IC₅₀≦5000 nM) as mentioned above, are listed below. Some of these compounds may bind to the sigma-1 and/or the sigma-2 receptor. Preferably, these compounds are in form of a salt, a base or an acid. Also preferably, the salts/bases/acids indicated in the list are to be understood as being exemplary and therefore may respresent any salt, base or acid of the compound.

| | |
|---|---|
| (−)-Cyanopindolol hemifumarate | (−)-SPARTEINE SULFATE |
| | PENTAHYDRATE |
| (+)-HIMBACINE | (2-Dibutylamino-Ethyl)-Carbamic Acid |
| | 2-(4-Benzofuran-2-Ylmethyl- |
| | Piperazin-1-Yl)-Ethyl Ester |
| (4-[1,2,3]Thiadiazol-4-Yl-Benzyl)- | (S)-Methamphetamine HCl |
| Carbamic Acid 1-(3-Methoxy-2-Nitro- | |
| Benzyl)-Piperidin-3-Ylmethyl Ester | |
| [1-(9-Ethyl-9H-Carbazol-3-Ylmethyl)- | [1-(9-Ethyl-9H-Carbazol-3-Ylmethyl)- |
| Pyrrolidin-3-Yl]-Carbamic Acid 1-(3- | Pyrrolidin-3-Yl]-Carbamic Acid 2- |
| Benzyloxy-4-Methoxy-Benzyl)-Piperidin- | (Tert-Butoxycarbonyl-Naphthalen-1- |
| 3-Ylmethyl Ester | Ylmethyl-Amino)-Ethyl Ester |
| [4-(4-Ethyl-3,5-Dimethyl-Pyrazol-1-Yl)- | 1-(1,2-Diphenylethyl)Piperidine |
| Phenyl]-[4-(3-Phenyl-Allyl)-Piperazin-1- | Maleate, (+/−) |
| Yl]-Methanone | |
| 1-(1-Naphthyl)Piperazine HCl | 1-(3-Chlorophenyl)Piperazine HCl |
| 1-(4-Bromo-Benzenesulfonyl)-4-(2-Tert- | 2-(2-{[1-(3-Chloro-Benzyl)-Pyrrolidin- |
| Butylsulfanyl-Benzyl)-Piperazine | 3-Yl]-Methyl-Carbamoyl}-2-Methyl- |
| | Propyl)-4,6-Dimethyl-Benzoic Acid |
| 2-Chloro-11-(4- | 3,3′-Diethylthiacarbocyanine Iodide |
| Methylpiperazino)Dibenz[B, F]Oxepin Maleate | |
| 3-Mercapto-2-Methylpropanoic Acid | 3-Quinuclidinyl Benzilate |
| 1,2-Diphenylethylamine Salt | |
| 3-Tropanyl-3,5-Dichlorobenzoate | 3-Tropanyl-Indole-3-Carboxylate HCl |
| 4-(1H-Indol-4-Yl)-Piperazine-1- | 4-(2-Tert-Butylsulfanyl-Benzyl)- |
| Carboxylic Acid 2-(5-Bromo-2-Ethoxy- | Piperazine-1-Carboxylic Acid 2- |
| Phenylamino)-Cyclohexylmethyl Ester | Thiophen-2-Yl-Ethyl Ester |
| 4-(3,5-Dimethoxy-Phenyl)-Piperazine-1- | 4-(3-Nitro-5-Sulfamoyl-Thiophen-2- |
| Carboxylic Acid 1-(2-Fluoro-Benzyl)- | Yl)-Piperazine-1-Carboxylic Acid 1-(2- |
| Piperidin-2-Ylmethyl Ester | Fluoro-5-Methoxy-Benzyl)-Piperidin-3- |
| | Ylmethyl Ester |
| 4-(4-Fluorobenzoyl)-1-(4- | 4-(5-Trifluoromethyl-Pyridin-2-Yl)- |
| Phenylbutyl)Piperidine Oxalate | Piperazine-1-Carboxylic Acid Pent-2- |
| | Ynyl Ester |
| 4,4′-Bis[4-(P-Chlorophenyl)-4- | 4-[1-(4-Chlorobenzyl)-4- |
| Hydroxypiperidino]Butyrophenone | (benzylpiperidin-4-yl]-2-hydroxy-4- |
| | oxobut-2-enoic acid |
| 4-Bromo-N-[1-(9-Ethyl-9H-Carbazol-3- | 4′-Chloro-3-Alpha- |
| Ylmethyl)-Pyrrolidin-3-Yl]-2- | (Diphenylmethoxy)Tropane HCl |
| Trifluoromethoxy-Benzenesulfonamide | |
| 4-Furan-2-Ylmethyl-Piperazine-1- | 4-Methoxy-N-[1-(7-Methoxy- |
| Carboxylic Acid 2-{4-[3-(2- | Benzo[1,3]Dioxol-5-Ylmethyl)- |
| Trifluoromethyl-Phenothiazin-10-Yl)- | Pyrrolidin-3-Yl]-Benzenesulfonamide |
| Propyl]-Piperazin-1-Yl}-Ethyl Ester | |
| 5-(N-Ethyl-N-Isopropyl)-Amiloride | 7-Hydroxy-DPAT HBr, (±)- |
| 8-Hydroxy-DPAT HBr, (R)-(+)- | 8-Hydroxy-DPAT HBr, S(−)- |
| 9-[4-({[4′-(trifluoromethyl)-1,1′-biphenyl- | Acepromazine Maleate |
| 2-yl]carbonyl}amino)piperidin-1-yl]-N- | |
| (2,2,2-trifluoroethyl)-9H-fluorene-9- | |
| carboxamide | |
| Acetophenazine Maleate | Acrinol |
| Ajmaline | Alaproclate HCl |
| Aloe-Emodin | Alprenolol D-Tartrate Salt Hydrate |
| Alprenolol HCl | AMI-193 |
| Aminobenztropine | Amiodarone HCl |
| Amodiaquine HCl | Amorolfine HCl |
| Amoxapine | Anileridine HCl |
| Anisotropine Methylbromide | Anpirtoline |
| ARC 239 DiHCl | Astemizole |
| Auramine O HCl | Azaperone |
| Azatadine Maleate | Azelastine HCl |
| Bamethan sulfate | BD 1008 DiHBr |
| BD-1047 | BD-1063 |
| Benextramine TetraHCl | Benfluorex HCl |
| Benidipine HCl | Benoxathian HCl |
| Benoxinate HCl | Benperidol |
| Benproperine Phosphate | Benzododecinium bromide |
| Benzphetamine HCl | Benztropine Mesylate |
| Benzydamine HCl | Bephenium Hydroxynaphthoate |
| Bepridil HCl | Berberine chloride |
| Betaxolol HCl | Bifemelane |
| BMY 7378 DiHCl | Bopindolol Malonate |
| BP 554 Maleate | Bromhexine HCl |
| Bromodiphenhydramine HCl | Bromperidol |
| Brompheniramine Maleate | BTCP HCl |
| Buclizine HCl | Buflomedil HCl |
| Bupropion HCl | Buspirone HCl |
| Butacaine Sulfate | Butaclamol HCl, (±)- |
| Butenafine HCl | Butoconazole Nitrate |
| BW 723C86 HCl | Carbetapentane Citrate |
| Carbinoxamine Maleate | Carpipramine DiHCl DiH2O |
| Carvedilol | Cephapirin Benzathine |
| CGS-12066A Maleate | Chloroprocaine HCl |
| Chloroquine Phosphate | Chlorpheniramine Maleate |
| Chlorphenoxamine HCl | Chlorpromazine HCl |
| Chlorprothixene | Cinanserin HCl |
| Cinnarizine | Cirazoline HCl |
| Cis-(+/−)-N-Methyl-N-[2-(3,4- | Cis(Z)-Flupentixol DiHCl |
| Dichlorophenyl)Ethyl]-2-(1- | |
| Pyrrolidinyl)Cyclohexamine DiHBr | |
| Cisapride Hydrate | Citalopram HBr |
| Clebopride Maleate Salt | Clemastine Fumarate |
| Clemizole HCl | Clenbuterol HCl |
| Clidinium Bromide | Clobenpropit 2HBr |
| Clofazimine | Clofilium Tosylate |
| Clomiphene Citrate | Clomiphene Related Compound A |
| Clomipramine | Cloperastine HCl |
| Clorgyline HCl | Clozapine |
| CONESSINE | Cyclizine |
| Cyclobenzaprine HCl | Cycloheximide |
| Cyproheptadine HCl | Darrow Red HCl |
| Demecarium Bromide | Denatonium Benzoate |
| Deptropine Citrate | Desloratadine |
| Dexbrompheniramine Maleate | Dexchlorpheniramine Maleate |
| Dexfenfluramine HCl | Dibucaine HCl |
| Dicyclomine HCl | Diethylpropion HCl |
| Dimethisoquin HCl | Dimetindene Maleate |
| Diphemanil Methylsulfate | Diphenidol HCl |
| Diphenoxylate HCl | Diphenylpyraline HCl |
| Dipropyldopamine HBr | Dobutamine HCl |
| Donepezil HCl | Doxepin HCl |
| Droperidol | Duloxetine |
| Dyclonine HCl | Ebastine |
| Econazole Nitrate | Epinastine HCl |
| Ethaverine HCl | Ethopropazine HCl |
| Eticlopride HCl, S(−)- | Etofenamate |
| Etonitazenyl Isothiocyanate | Femoxetine HCl |
| Fenfluramine HCl | Fentanyl Citrate |
| Fenticonazole Nitrate | Fipexide HCl |
| Flavoxate HCl | Flunarizine diHCl |
| Fluoxetine Related Compound B | Fluperlapine |
| Fluphenazine Decanoate DiHCl | Fluphenazine Enanthate DiHCl |
| Fluphenazine HCl | Fluphenazine N-Mustard DiHCl |
| Flurazepam Related Compound C | Fluspirilene |
| Fluvoxamine Maleate | GBR 12783 DiHCl |
| GBR 12909 DiHCl | GBR 13069 DiHCl |
| GBR-12935 DiHCl | GR 89696 Fumarate |
| Guanabenz Acetate | Guanadrel Sulfate |
| Guanethidine Sulfate | Halofantrine HCl |
| Haloperidol | HEAT HCl |
| Hexylcaine HCl | Hycanthone |
| Hydroxychloroquine Sulfate | Hydroxyzine HCl |
| Hyoscyamine Sulfate | IBZM, S(−)- |
| ICI-199,441 HCl | Ifenprodil Tartrate |
| Imipramine HCl | Indatraline HCl |
| Iofetamine HCl | Irinotecan HCl |
| Isamoltane Hemifumarate | Isopromethazine HCl |
| Isoxsuprine HCl | Ketanserin L-Tartrate |
| Ketoconazole | Ketotifen Fumarate Salt |
| L-693,403 Maleate | L-741,626 |
| L-741,742 HCl | L-745,870 TriHCl |
| Labetalol HCl | Levetimide HCl, R(−) |
| Levobunolol HCl | Lidoflazine |
| Lisuride Hydrogen Maleate, R(+)- | Lobeline HCl |
| lomerizine diHCl | Loperamide HCl |
| Loxapine Succinate | LY-53,857 Maleate |
| Maprotiline HCl | Mazindol |
| MDL 12,330A HCl | Mebhydroline 1,5- |
| | naphthalendisulfonate Salt |
| Meclizine HCl | Mefloquine HCl |
| Meprylcaine HCl | Mesoridazine Besylate |
| Metaphit Methanesulfonate | Metergoline |
| Methantheline Bromide | Methdilazine |
| Methiothepin Mesylate | Methixene HCl |
| Methoctramine | Methotrimeprazine Maleate |
| Methylene Violet 3Rax HCl | Metipranolol |
| Mexiletine HCl | Mianserin HCl |
| Miconazole | ML-9 HCl |
| Morantel Hydrogen L-Tartrate | MR 16728 HCl |
| N-(2-Chloroethyl)-N-Ethyl-2- | N′-[2-(Benzo[1,2,5]Thiadiazole-4- |
| Bromobenzylamine HCl | Sulfonylamino)-Acetyl]- |
| | Hydrazinecarboxylic Acid 2-(2-{4-[(4- |
| | Chloro-Phenyl)-Phenyl-Methyl]- |
| | Piperazin-1-Yl}-Ethoxy)-Ethyl Ester |
| Nafronyl Oxalate Salt | Naftifine |
| Naftopidil diHCl | Naltriben Mesylate |
| NAN-190 HBr | NE-100 |
| Nefazodone | Nefopam HCl |
| Nicardipine HCl | Nicergoline |
| Niguldipine HCl, (+/−)- | Nisoxetine HCl |
| Nortriptyline HCl | Nylidrin HCl |
| Octoclothepin Maleate, (±)- | Orphenadrine Citrate |
| Oxamniquine | Oxamniquine Related Compound A |
| Oxamniquine Related Compound B | Oxatomide |
| Oxiconazole Nitrate | Oxybutynin HCl |
| Panaxatriol | PAPP |
| Paroxetine | Paxilline |
| p-Chlorobenzhydrylpiperazine | Penbutolol Sulfate |
| Pentamidine Isethionate | Pentazocine, (±)- |
| Pergolide Methanesulfonate | Perhexiline Maleate Salt |
| Perospirone | Perphenazine |
| Perphenazine Sulfoxide | Phenamil Methanesulfonate |
| Phencyclidine HCl | Phenosafranin HCl |
| Phenoxybenzamine HCl | Phenyltoloxamine Citrate Salt |
| Piboserod | Pimozide |
| Pinacyanol Chloride | Pindobind, (+/−)- |
| Piperacetazine | Piperazine-1,4-Dicarboxylic Acid |
| | Benzyl Ester 2-[4-(4-Dimethylamino- |
| | Benzyl)-Piperazin-1-Yl]-Ethyl Ester |
| Piperidolate HCl | Pirenperone |
| PPHT HCl, (±)- | Pramoxine HCl |
| Prenylamine Lactate Salt | Pridinol Methanesulfonate Salt |
| Prochlorperazine Maleate | Procyclidine HCl |
| Proflavine Hemisulfate Salt | Progesterone |
| Promazine HCl | Promethazine HCl |
| Propafenone HCl | Proparacaine HCl |
| Propericyazine | Propiomazine |
| Propranolol HCl | Protokylol |
| Protriptyline HCl | Pyrilamine Maleate |
| Pyrimethamine | Pyrrolidine-1,2-Dicarboxylic Acid 1-[1- |
| | (4-Allyloxy-Benzyl)-Piperidin-2- |
| | Ylmethyl] Ester 2-Benzyl Ester |
| Pyrvinium Pamoate | Quetiapine Fumarate |
| Quinacrine HCl | Quinaldine Red |
| Quipazine Dimaleate | Quipazine, 6-Nitro-, Maleate |
| Raloxifene | Rimantadine HCl |
| Risperidone | Ritanserin |
| Ritodrine HCl | RS 23597-190 HCl |
| RS 67333 HCl | RS 67506 HCl |
| Safranin O HCl | Salmeterol |
| SB203186 | SCH-23390 HCl, R(+)- |
| Sertaconazole Nitrate | Sertindole |
| Sertraline | Sibutramine HCl |
| SKF-525A HCl | SKF-96365 HCl |
| SNC 121 | Spiperone HCl |
| Sufentanil | T-226296 |
| Tamoxifen Citrate | Tamsulosin HCl |
| Tegaserod Maleate | Terbinafine HCl |
| Terconazole | Terfenadine |
| Terfenadine Related Compound A | Tetracaine HCl |
| Tetrindole Mesylate | Thiethylperazine Malate |
| Thioperamide Maleate | Thioproperazine |
| Thioridazine | Thiothixene |
| Thiothixene, (E)- | Thonzonium Bromide |
| Tioconazole Related Compound A | TMB-8 HCl |
| Tolterodine L-Tartrate | Toremifene Citrate |
| Tramazoline HCl | Trans-U-50488 Methanesulfonate, |
| | (±)- |
| Trazodone HCl | Tridihexethyl Chloride |
| Trifluoperazine HCl | Trifluperidol HCl |
| Triflupromazine HCl | Trihexyphenidyl HCl |
| Trimebutine | Trimeprazine Hemi-L-Tartrate |
| Trimipramine Maleate | Tripelennamine HCl |
| Triprolidine HCl | Triprolidine HCl Z Isomer |
| Tropanyl 3,5-Dimethylbenzoate | Tropine 2-(4- |
| | Chlorophenoxy)Butanoate, Maleate |
| U-50488 HCl, (−)- | U-62066 |
| UH 232 Maleate, (+)- | Vecuronium Bromide |
| Verapamil HCl | Verapamil Related Compound B |
| Vesamicol HCl | Vinpocetine |
| W-7 HCl | WB-4101 HCl |
| Xylazine | Xylometazoline HCl |

Another aspect of the present invention relates to BD-1063 and its derivatives, a compound binding to the sigma receptor, and its use for the production of a medicament for the treatment of mechanical allodynia.

The synthesis of BD-1063 and compounds structurally related (mostly those covered under formula IB) is described in detail in de Costa et al. (1993), J. Med. Chem. 36(16): 2311-2320 (referred to as compound 4 in Scheme I; p. 2312) included here by reference. Accordingly the synthesis of the compound whose use is claimed in this invention is known and the compound thus is available to those skilled in the art, starting from the given information working analogously if necessary.

Compounds according to general formula IB have been shown to be sigma ligands according to the above mentioned definition.

Therefore, another aspect of the present invention is the use of a compound of general formula IB

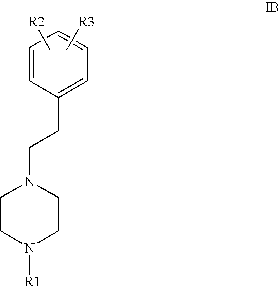

wherein
R1 is selected from C₁₋₆-Alkyl, saturated or unsaturated, substituted or not substituted, branched or not branched;
R2 and R3 are independently of each other selected from H; C₁₋₆-Alkyl, saturated or unsaturated, substituted or not substituted, branched or not branched; Halogen, O—C₁₋₆-Alkyl, saturated or unsaturated, substituted or not substituted, branched or not branched; in the production of a drug to treat mechanical allodynia.

In a preferred embodiment of the invention the compound used is a compound according to general formula IB, wherein R1 is selected from C₁₋₆-Alkyl, saturated or unsaturated, branched or not branched, unsubstituted or substituted with F, Cl, Br, I, NH₂, SH or OH.

In a preferred embodiment of the invention the compound used is a compound according to general formula IB, wherein R2 and R3 are independently of each other selected from H; C₁₋₆Alkyl, saturated or unsaturated, branched or not branched, unsubstituted or substituted with F, Cl, Br, I, NH₂, SH or OH; Halogen; O-C₁₋₆-Alkyl, saturated or unsaturated, branched or not branched, unsubstituted or substituted with F, Cl, Br, I, NH₂, SH or OH.

In a preferred embodiment of the invention the compound used is a compound according to general formula IB, wherein
R¹ is selected from C₁₋₄-Alkyl, saturated or unsaturated, substituted or not substituted, and branched or not branched;
preferably R¹ is selected from C₁₋₄-Alkyl, saturated, substituted or not substituted, and branched or not branched;
more preferably R¹ is selected from C₁₋₄-Alkyl, saturated, not substituted, and branched or not branched; namely CH₃, C₂H₅, C₃H₇, C₄H₉.

In a preferred embodiment of the invention the compound used is a compound according to general formula IB, wherein
R² and R³ are independently of each other selected from H; OH, SH, NH₂, C₁₋₄-Alkyl, saturated or unsaturated, substituted or not substituted, branched or not branched; F, Cl, Br, I; O—C₁₋₄-Alkyl, saturated or unsaturated, substituted or not substituted, branched or not branched;
preferably R² and R³ are independently of each other selected from H; OH, NH₂, C₁₋₄Alkyl, saturated, substituted or not substituted, and branched or not branched; F, Cl, Br, I; O—C₁₋₄-Alkyl, saturated, not substituted, and branched or not branched.

In a preferred embodiment of the invention the compound used is a compound according to general formula IB, wherein
R² and R³ are independently of each other selected from H, OH, NH₂, CH₃, C₂H₅, C₃H₇, C₄H₉, CF₃, CHF₂, Cl, F, Br, I, O—CH₃, O—C₂H₅, O—C₃H₇, O—C₄H₉;
preferably R² and R³ are independently of each other selected from H, F, Cl and CF₃.

Here it is also preferred if R² and R³ are in 3′ and 4′ position on the phenyl ring.

In a highly preferred embodiment of the invention the compound according to general formula IB used is 1-(3,4-dichlorophenethyl)-4-methylpiperazine, optionally in the form of its racemate, pure stereoisomers, especially enantiomers or diastereomers or in the form of mixtures of stereoisomers, especially enantiomers or diastereomers, in any suitable ratio; in the form described or in form of an acid or base or in form of a salt, especially a physiologically acceptable salt, or in form of a solvate, especially a hydrate.

Unless otherwise stated, the compound of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or ¹⁵N-enriched nitrogen are within the scope of this invention.

Another aspect of the present invention relates to BD-1047, a compound binding to the sigma receptor, and its use for the production of a medicament for the treatment of mechanical allodynia.

BD-1047 (N1-(3,4-dichlorophenethyl)-N1,N2,N2-trimethylethane-1,2-diamine/N-[2-(3,4-dichlorophenyl)ethyl]-N-methyl-2-(dimethylamino)-ethylamine) is, like BD-1063, commercially available and also a popular tool compound as sigma receptor ligands (K₁=0.9 nM for sigma-1). As according to U.S. 2003/0171347A1, BD-1047 shows no or only slight binding to a selection of other relevant receptors (page 7, table2 and page 8, table 3).

The synthesis of BD-1047 is described in detail in de Costa et al. (1992), J. Med Chem. 35, 38-47, as well as WO92/22279 A1 showing compounds related to CNS disorders, where BD-1047 is described as a truncated version (compound 10 in Scheme 2, p. 17), included herein by reference.

Another aspect of the invention is the use of BD-1047 and/or at least one compound of general formula IB as defined above for the production of a medicament for the treatment of neuropathic pain.

In the context of the whole invention, alkyl and cycloalkyl radicals are understood as meaning saturated and unsaturated (but not aromatic), branched, unbranched and cyclic hydrocarbons, which can be unsubstituted or mono- or polysubstituted. In these radicals, C₁₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C 16-, C17- or C18-alkyl. Furthermore, C₃₋₄-cycloalkyl represents C3- or C4-cycloalkyl, C₃₋₅-cycloalkyl represents C3-, C4- or C5-cycloalkyl, C₃₋₈-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆ -cycloalkyl represents C5- or C6-cycloalkyl and C₅₋₇-cycloalkyl represents C5-, C6- or C7-cycloalkyl. In respect of cycloalkyl, the term also includes saturated cycloalkyls in which one or 2 carbon atoms are replaced by a heteroatom, S, N or O. However, mono- or polyunsaturated, preferably monounsaturated, cycloalkyls without a heteroatom in the ring also in particular fall under the term cycloalkyl as long as the cycloalkyl is not an aromatic system. The alkyl and cycloalkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl, and also adamantyl, (if substituted also CHF₂, CF₃ or CH₂OH) as well as pyrazolinone, oxopyrazolinone, [1,4]-dioxane or dioxolane.

In the context of the whole invention, in connection with alkyl and cycloalkyl—unless expressly defined otherwise—the term substituted in the context of this invention is understood as meaning replacement of at least one hydrogen radical by F, Cl, Br, I, NH₂, SH or OH, “polysubstituted” radicals being understood as meaning that the replacement takes effect both on different and on the same atoms several times with the same or different substituents, for example three times on the same C atom, as in the case of CF₃, or at different places, as in the case of —CH(OH)—CH=CH—CHCl₂. Particularly preferred substituents here are F, Cl and OH. In respect of cycloalkyl, the hydrogen radical can also be replaced by OC₁₋₃-alkyl or C₁₋₃-alkyl (in each case mono- or polysubstituted or unsubstituted), in particular methyl, ethyl, n-propyl, i-propyl, CF₃, methoxy or ethoxy.

In a preferred embodiment of the present invention, with respect to compounds of general formula IB, R1, R2 and/or R3 are at least optionally substituted with F, Cl, Br, I, NH₂, SH or OH.

Referring to the whole invention, the term (CH₂)₃₋₆ is to be understood as meaning —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, (CH₂)₁₋₄ is to be understood as meaning —CH₂—, —CH₂—CH₂—, —CH₂—CH₂—CH₂— and —CH₂—CH₂—CH₂—CH₂—, (CH₂)₄₋₅ is to be understood as meaning —CH₂—CH₂—CH₂—CH₂— and —CH₂—CH₂—CH₂—CH₂—CH₂—, etc.

An aryl radical is understood in the whole invention as meaning ring systems with at least one aromatic ring but without heteroatoms even in only one of the rings. Examples are phenyl, naphthyl, fluoranthenyl, fluorenyl, tetralinyl or indanyl, in particular 9H-fluorenyl or anthracenyl radicals, which can be unsubstituted or monosubstituted or polysubstituted.

Additionally, in the context of the whole invention, a heteroaryl radical is understood as meaning heterocyclic ring systems which have at least one unsaturated ring and can contain one or more heteroatoms from the group consisting of nitrogen, oxygen and/or sulfur and can also be mono- or polysubstituted. Examples which may be mentioned from the group of heteroaryls are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole and quinazoline.

In addition, referring to the whole invention, in connection with aryl and heteroaryl, substituted is understood as meaning substitution of the aryl or heteroaryl by R, OR, a halogen, preferably F and/or Cl, a CF₃, a CN, an NO₂, an NRR, a C₁₋₆-alkyl (saturated), a C₁₋₆-alkoxy, a C₃₋₈-cycloalkoxy, a C₃₋₈-cycloalkyl or a C₂₋₆-alkylene.

The term “salt” is to be understood as meaning any form of the active compound according to the invention in which this assumes an ionic form or is charged and is coupled with a counter-ion (a cation or anion) or is in solution. By this are also to be understood complexes of the active compound with other molecules and ions, in particular complexes which are complexed via ionic interactions.

The term “physiologically acceptable salt” is understood in particular, in the context of this invention, as salt (as defined above) formed either with a physiologically tolerated acid, that is to say salts of the particular active compound with inorganic or organic acids which are physiologically tolerated—especially if used on humans and/or mammals—or with at least one, preferably inorganic, cation which are physiologically tolerated—especially if used on humans and/or mammals. Examples of physiologically tolerated salts of particular acids are salts of: hydrochloric acid, hydrobromic acid, sulfuric acid, hydrobromide, monohydrobromide, monohydrochloride or hydrochloride, methiodide, methanesulfonic acid, formic acid, acetic acid, oxalic acid, succinic acid, malic acid, tartaric acid, mandelic acid, fumaric acid, lactic acid, citric acid, glutamic acid, hippuric acid picric acid and/or aspartic acid. Examples of physiologically tolerated salts of particular bases are salts of alkali metals and alkaline earth metals and with NH₄.

The term “solvate” according to this invention is to be understood as meaning any form of the active compound according to the invention in which this compound has attached to it via non-covalent binding another molecule (most likely a polar solvent) especially including hydrates and alcoholates, e.g. methanolate.

According to the IASP “allodynia” is defined as “a pain due to a stimulus which does not normally provoke pain” (IASP, Classification of chronic pain, 2^{nd} Edition, IASP Press (2002), 210). Even though the symptoms of allodynia are most likely associated as symptoms of neuropathic pain this is not necessarily the case so that there are symptoms of allodynia not connected to neuropathic pain though rendering mechanical allodynia in some areas broader then neuropathic pain.

Mechanical allodynia is a form of allodynia where mechanical stimuli cause the painful sensation in contrast to thermal allodynia where the painful sensation comes from a thermal stimulus (e.g. heat).

“Neuropathic pain” is defined by the IASP as “pain initiated or caused by a primary lesion or dysfunction in the nervous system” (IASP, Classification of chronic pain, 2nd Edition, IASP Press (2002), 210). For the purpose of this invention included under this heading or to be treated as synonymous is “Neurogenic Pain” which is defined by the IASP as “pain initiated or caused by a primary lesion, dysfunction or transitory perturbation in the peripheral or central nervous system”.

The IASP draws the following difference between “allodynia”, “hyperalgesia” and “hyperpathia” (IASP, Classification of chronic pain, 2^{nd} Edition, IASP Press (2002), 212):

| | | |
|---|---|---|
| Allodynia | Lowered threshold | Stimulus and response |
| | | mode differ |
| Hyperalgesia | Increased response | Stimulus and response |
| | | rate are the same |
| Hyperpathia | Raised threshold; | Stimulus and response |
| | Increased response | rate may be the same or |
| | | different |

In another preferred embodiment of the use according to the invention the mechanical allodynia derived from central pain.

In another preferred embodiment of the use according to the invention the mechanical allodynia derived from peripheral pain.

In another preferred embodiment of the invention the mechanical allodynia is derived from neuropathic pain.

In a very preferred embodiment of the invention the compound binding to the sigma receptor used is acting on the sigma receptor as an antagonist.

In a very preferred embodiment of the invention the compound binding to the sigma receptor used is acting on the sigma receptor as an inverse agonist.

In a very preferred embodiment of the invention the compound binding to the sigma receptor used is acting on the sigma receptor as a partial antagonist.

In another possible embodiment of the invention the compound binding to the sigma receptor used is acting on the sigma receptor as an agonist.

In another embodiment of the invention the compound binding to the sigma receptor used is acting on the sigma receptor as a mixed agonist/antagonist, a partial agonist or a partial antagonist.

In another embodiment of the invention the sigma receptor to which the “compound binding to the sigma receptor” is binding to is the sigma-1 receptor. Under this embodiment “Compound/s binding to the sigma receptor” as used in this application is/are defined as having an IC50 value ≦5000 nM, more preferably ≦1000 nM, more preferably ≦500 nM. More preferably, the IC₅₀ value is ≦250 nM. More preferably, the IC₅₀ value is ≦100 nM. Most preferably, the IC₅₀ value is ≦50 nM. Additionally, the wording “Compound/s binding to the sigma receptor”, as used in the present application is defined as having at least ≧50% displacement using 10 mM radioligand specific for the sigma receptor (e.g. preferably ¹H-pentazocine) whereby the sigma recepor may be any sigma receptor subtype.

In another preferred embodiment of the invention, the compound binding to the sigma receptor, as defined above, has an IC₅₀ value of ≦1000 nM.

In another preferred embodiment of the invention, the compound binding to the sigma receptor, as defined above, has an IC₅₀ value of ≦500 nM.

In another preferred embodiment of the invention, the compound binding to the sigma receptor, as defined above, has an IC₅₀ value of ≦250 nM.

In another preferred embodiment of the invention, the compound binding to the sigma receptor, as defined above, has an IC₅₀ value of ≦100 nM.

In another preferred embodiment of the invention, the compound binding to the sigma receptor, as defined above, has an IC₅₀ value of ≦50 nM.

Most preferably, “compounds highly specific for the sigma receptor” are defined as being “Compound/s binding to the sigma receptor”, as defined above, having an IC₅₀ value of ≦100 nM.

In a highly preferred embodiment of the present invention, the compound binding to the sigma receptor as defined above, is binding to the sigma-1 receptor subtype.

In another possible aspect of the invention, the compound binding to the sigma receptor as defined above, may bind to the sigma-2 receptor subtype.

In human therapeutics, the dose administered can be quite low depending on the route of administration and is well known in the art because sigma compounds are known therapeutics.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

Any medicament according to the invention contains the active ingredient as well as optionally at least one auxiliary material and/or additive and/or optionally another active ingredient.

The auxiliary material and/or additive can be specifically selected from conserving agents, emulsifiers and/or carriers for parenteral application. The selection of these auxiliary materials and/or additives and of the amounts to be used depends upon how the pharmaceutical composition is to be applied. Examples include here especially parenteral like intravenous subcutaneous or intramuscular application formulations but which could also be used for other administration routes.

Routes of administration preferably include intramuscular injection, intraveneous injection, subcutaneous injection, sublingual, bucal, patch through skin, oral ingestion, implantable osmotic pump, collagen implants, aerosols or suppository.

Included in this invention are especially also methods of treatments of a patient or a mammal, including men, suffering from mechanical allodynia using compounds binding to the sigma receptor.

The examples and figures in the following section describing pharmacological trials are merely illustrative and the invention cannot be considered in any way as being restricted to these applications.

### EXAMPLES

### Example 1

### Von Frey-Model

The von Frey model is a model for allodynia, stimulated mechanically (mechanical allodynia).

Interest of the model:
The injection of capsaicin to experimental animals produces acute pain followed by allodynia
The mechanisms involved in capsaicin-induced acute pain and allodynia are relatively well known (mainly activation of peripheral nociceptors and sensitization of spinal cord neurons, respectively)

### Hypothesis

Capsaicin-induced allodynia is due to the release in the spinal cord of several substances including excitatory aminoacids (EA). Since sigma ligands modulate the effect of EA they would also modulate capsaicin-induced allodynia

FIG. 1) shows the test protocol for all tests with von Frey filaments. After habituation mice were according to FIG. 1 first treated with the test-compound (or not in controls). Then capsaicin (1% DMSO) is injected into their paw resulting in developing pain in the effected paw. The effected paw is then treated with a mechanical stimulus and the latency time before the paw is withdrawn is measured.

### Example 2

### Effect of NE-100 in the von Frey-Model

NE-100 is a well known compound with high affinity to the sigma receptor, more specifically a known specific inhibitor of Sigma 1 (J Med Chem 1999, 42(19): 3965). This pharmacological test showed the effect of NE-100 a specific sigma receptor inhibitor in the von-frey model described in example 1, a model of allodynia.

As shown in FIG. 2a) there is a dose dependency of the treatment with NE-100 showing analgesia in capsaicin-induced allodynia.

As demonstrated in FIG. 2b) the treatment with NE-100 is effective specifically in allodynia or mechanical allodynia and not general pain as shown by the different efficacy depending on the force of the von-Frey filaments with 0.5 g being typically in the range of allodynia and 4 g clearly being in the general pain field.

Further as shown in FIG. 2c) there is clear evidence that the effect of the treatment with NE-100 is clearly connected to its sigma inhibitor activity, as PRE-084 is a well known sigma receptor agonist counteracting the effect of NE-100.

### Example 3

### Effect of Antisense ODN Against the Sigma Receptor in the von Frey-Model

2 well known antisense Oligodesoxynucleotides (ODN) against the sigma 1 receptor (King et al. (1997) Eur J Pharmacol 331:R5-R6 and Ueda et al. (2001) J. Pharmacol. Exp. Ther. 298, 703-710) were synthesized and according to the protocol shown in FIG. 3a) given on 4 consecutive days i.c.v. followed by a wash-out period and von-Frey tests according to example 1.

As can be seen in FIG. 3b) both antisense ODNs show a strong effect on day one after treatment with mismatches not having any significant effect. This effect is washed out after 7 days as can be expected from antisense ODN.

The effectiveness and dose dependency is demonstrated in FIG. 3c). Mismatches do not have any significant effect.

Further as demonstrated in FIG. 3d) the treatment with the two known antisense ODNs is effective specifically in allodynia or mechanical allodynia and not general pain as shown by the different efficacy depending on the force of the von-Frey filaments with 0.5 g being typically in the range of allodynia and 4 g clearly being in the general pain field.

### Example 4

### Effect of the von Frey-Model on KO Mice

KO mice lacking the sigma 1 receptor were prepared according to WO 2004/52092 and tested in comparison to wild-type mice in the von-Frey model. As demonstrated in FIG. 4) KO-Mice not having the sigma (1) receptor (knock-out mice) are not susceptible anymore to the allodynia inducing effects of capsaicin independent of the dose given compared to wild-type mice (called wild-type mice). This is clearly demonstrating the truth of the role of sigma receptors in allodynia and strengthens the claim to the role of all compounds binding to the sigma-receptor in allodynia.

### Example 5

### Effects of BD-1063 in the von Frey Model (Mechanical Allodynia) in Mice Exposed to the Sciatic Nerve Injury

### Surgery

The partial sciatic nerve ligation model was used to induce neuropathic pain (Malmberg and Basbaum, Pain 76: 215-222, 1998). This model consists of injury to the sciatic nerve at mid-to high level. Briefly, mice were anaesthetized with halothane (induction: 3%; surgery: 1%) and the common sciatic nerve was exposed at the level of the mid-thigh of the right paw. In control animals (sham-operated mice), an identical dissection was performed on the right paw except that the sciatic nerve was not ligated (Control). Nociceptive sensitivity was then measured was quantified by measuring the hind paw withdrawal response to von Frey filament stimulation as mentioned above. Animals underwent surgery on day 0; 11 days after surgery BD-1063 was injected subcutaneously in one group and the responses to mechanical stimuli were determined by means of the von Frey model in this and the none-treated group (before surgery) a reference value was measured in order to determine a baseline value.

### FIGURES

FIG. 1) refers to example 1 and shows the test protocol for all tests with von Frey filaments.

FIG. 2 a to c) refer to example 2 and show the effect of NE-100 a specific sigma receptor inhibtor (IC₅₀=1.3 nM; J Med Chem 1999, 42(19): 3965) in a model of allodynia, especially mechanical allodynia.

FIG. 2a) shows the dose dependency of the treatment with NE-100 to show analgesia in capsaicin-induced allodynia.

FIG. 2b) demonstrates that the treatment with NE-100 is effective specifically-in mechanical allodynia and not general pain as shown by the different efficacy depending on the force of the von-Frey filaments with 0.5 g being typically in the range of allodynia and 4 g clearly being in the general pain field. A:Solvent, B:NE-100 [64 mg/kg]

FIG. 2c) proofs that the effect of the treatment with NE-100 is clearly connected to its sigma inhibitor activity, as PRE-084 is a well known sigma receptor agonist.

FIGS. 3 a to d) refer to example 3 and shows the effect of antisense ODNs against sigma (1) receptor.

FIG. 3a) shows the test protocol for Oligodesoxynucleotid (ODN) tests with von Frey filaments.

FIG. 3b) shows the influence of the wash-out period on the effect t of treatment with antisense ODN, with two known antisense ODN (by King et al. (1997) Eur J Pharmacol 331:R5-R6 and Ueda et al. (2001) J. Pharmacol. Exp. Ther. 298, 703-710) being used proving their highly significant effect on allodynia in the von-Frey model. Still after 7 days washout the effect is gone as has to be expected from antisense ODN. Mismatches do not have any significant effect. A:Saline 2 μl/mouse, B:ODN KING 10 μg/mouse, C:ODN UEDA 10 μg/mouse, D:Mismatch UEDA 10 μg/mouse

FIG. 3c) shows the effectiveness and dose dependency with two known antisense ODNs (by King et al. (1997) Eur J Pharmacol 331:R5-R6 and Ueda et al. (2001) J. Pharmacol. Exp. Ther. 298, 703-710) testing with von Frey filaments. Mismatches do not have any significant effect. A:ODN UEDA, B:Mismatch UEDA, C:ODN KING, D:Mismatch KING

FIG. 3d) demonstrates that the treatment with two known antisense ODNs is effective specifically in mechanical allodynia and not general pain as shown by the different efficacy depending on the force of the von-Frey filaments with 0.5 g being typically in the range of allodynia and 4 g clearly being in the general pain field. A:Saline 2 μl/mouse, B:ODN KING 10 μg/mouse, C:Mismatch KING 10 μg/mouse, D:ODN UEDA 10 μg/mouse, E:Mismatch UEDA 10 μg/mouse.

FIG. 4) refers to example 4 and demonstrates clearly that KO-Mice not having the sigma (1) receptor (called “mutantes”) are not susceptible anymore to the allodynia-inducing effects of capsaicin independent of the dose given compared to wild-type mice. This is clearly demonstrating the truth of the role of sigma receptors in allodynia and strengthens the claim to the role of all compounds binding to the sigma-receptor in allodynia. A:Wild-type mice, B:Knock-out mice.

FIG. 5) refers to example 5 and demonstrates the effects of BD-1063, a known antagonist (IC₅o=30 nM sigma- 1/800 nM sigma-2) in the von Frey model (mechanical allodynia) in mice exposed to the sciatic nerve injury. A:Baseline:Day 0, before surgery; B:Allodynic effects of sciatic nerve surgery on Day 11; C:Day 11 =BD-1063 (60mg/kg) subcutaneous administration. It can be clearly seen that BD-1063, being a specific sigma receptor antagonist, antagonizes mechanical allodynia in mice exposed to the (ipsilateral) sciatic nerve injury significantly. It can also be clearly demonstrated that the mechanical allodynia is attributed to the sigma-1 receptor since heterozygous mice only show half of the mechanical allodynic response. Knock-out mice do not develop mechanical allodynia.

FIG. 6) refers to example 6 and demonstrates the Effects of BD-1063, a known antagonist, in the von Frey model (mechanical allodynia) in sham-operated mice. A:Day 0=Baseline; before sciatic nerve surgery; B:Effects of sciatic nerve surgery; C:Administration of BD-1063 (60 mg/kg) on Day 11. This control experiments clearly shows that the effect shown in FIG. 5) is sigma-specific.

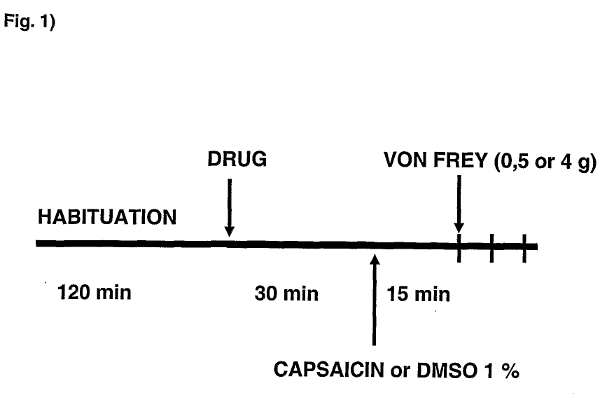

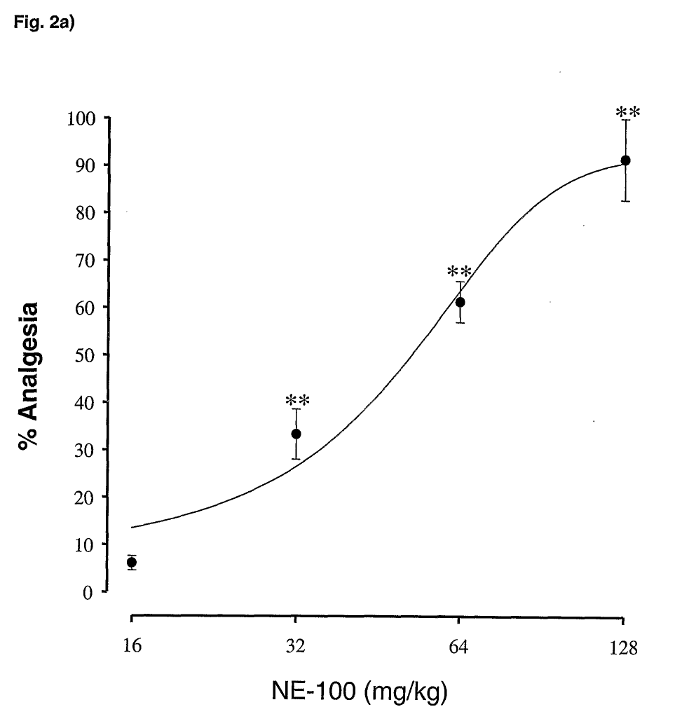

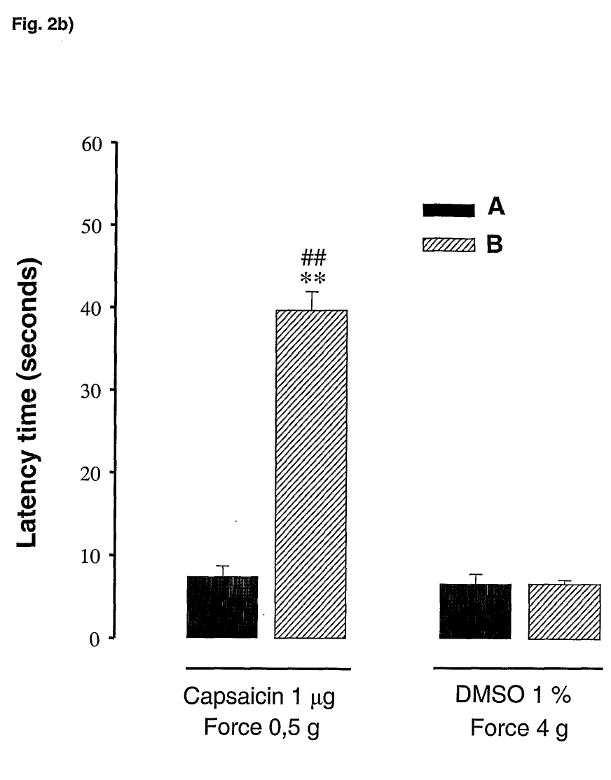

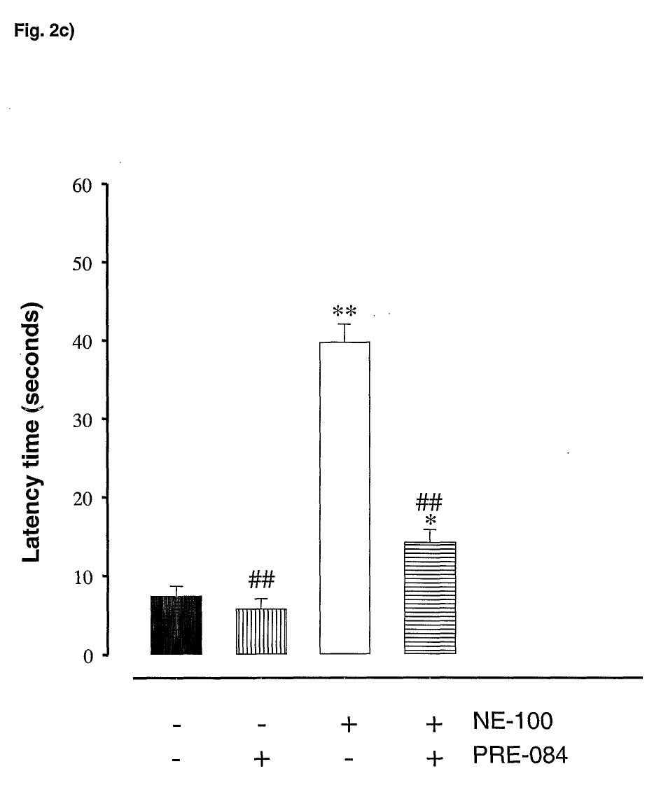

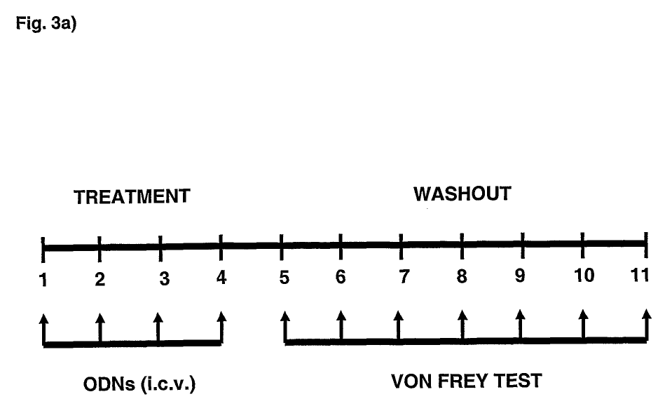

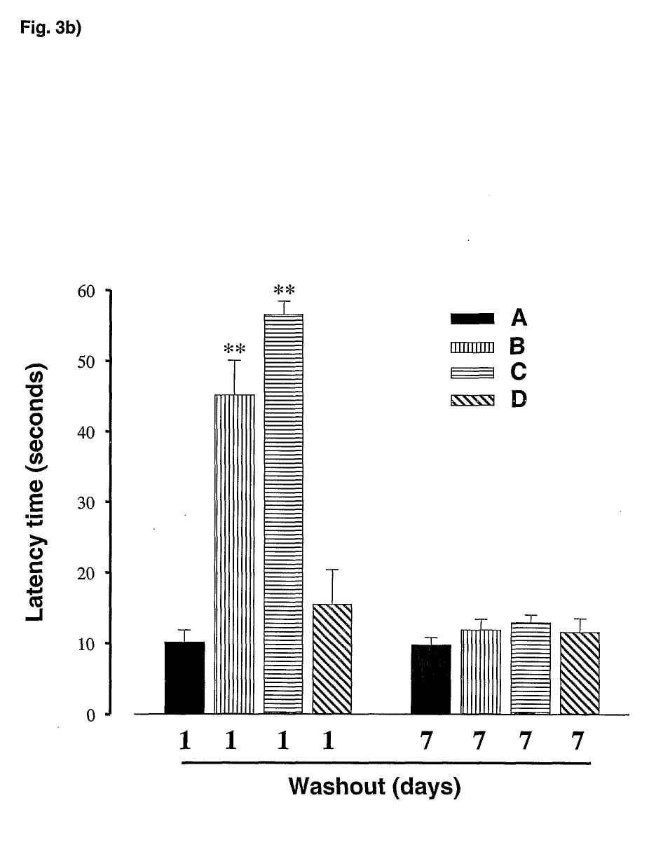

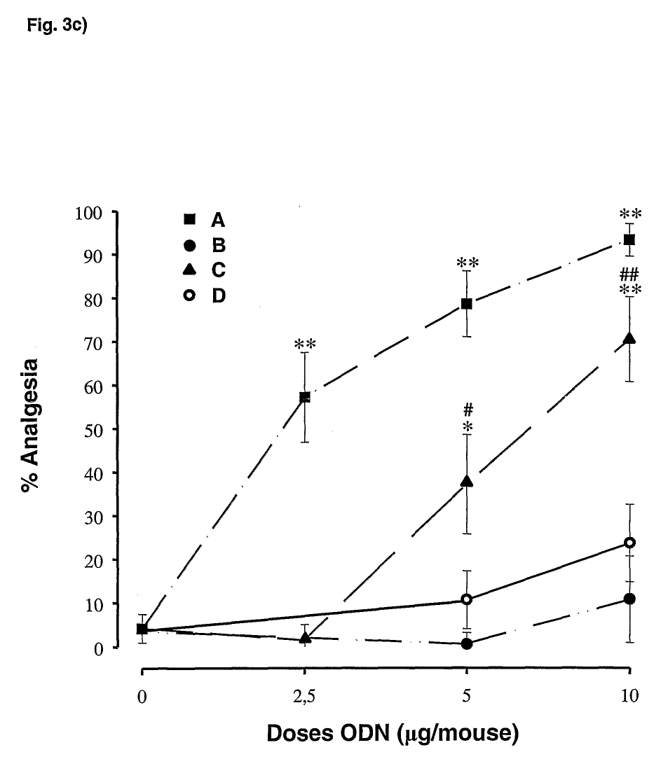

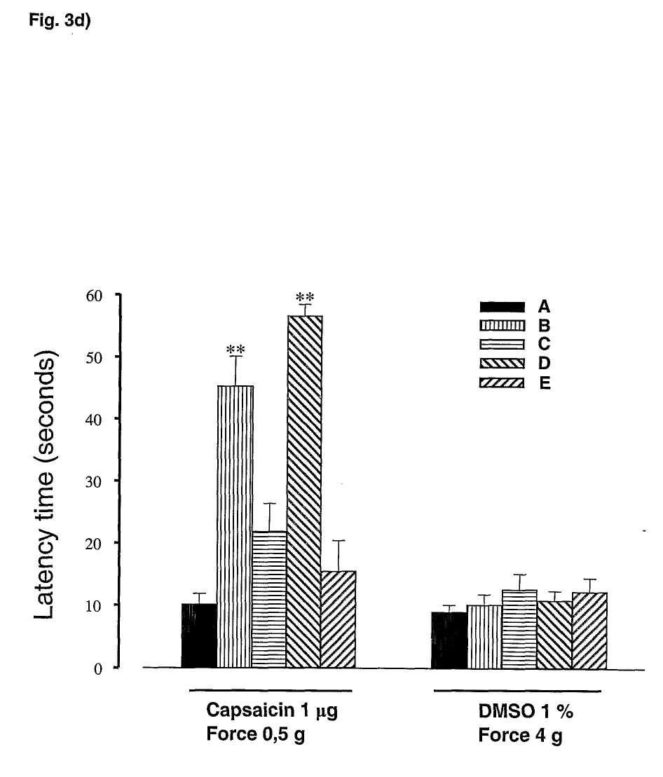

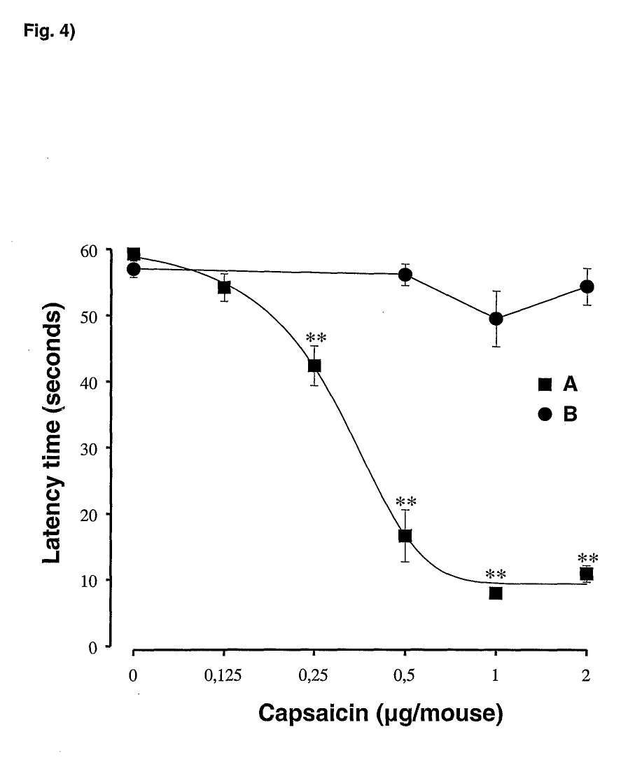

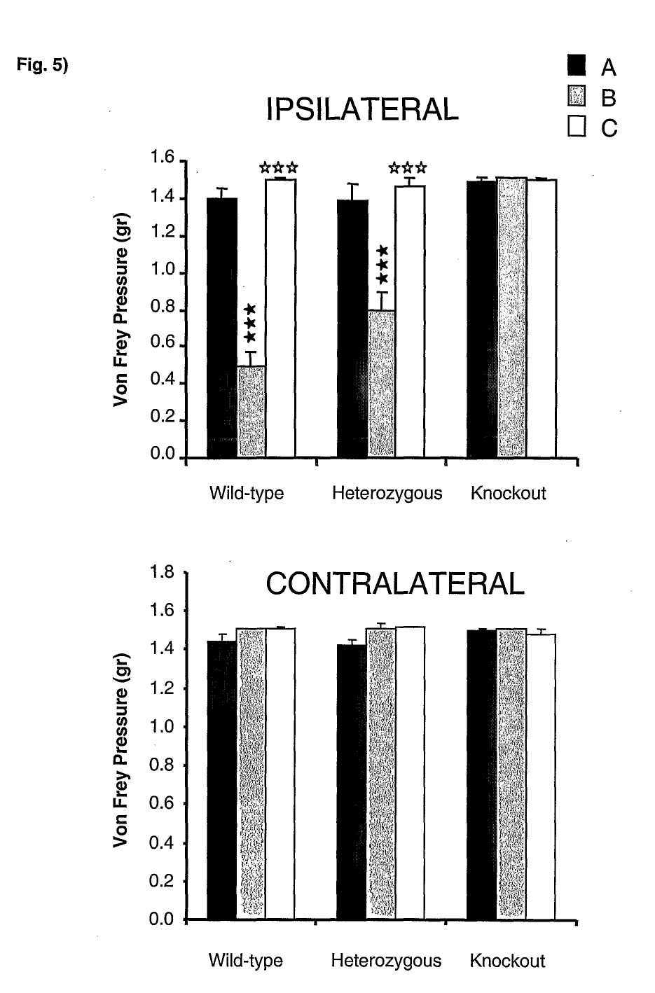

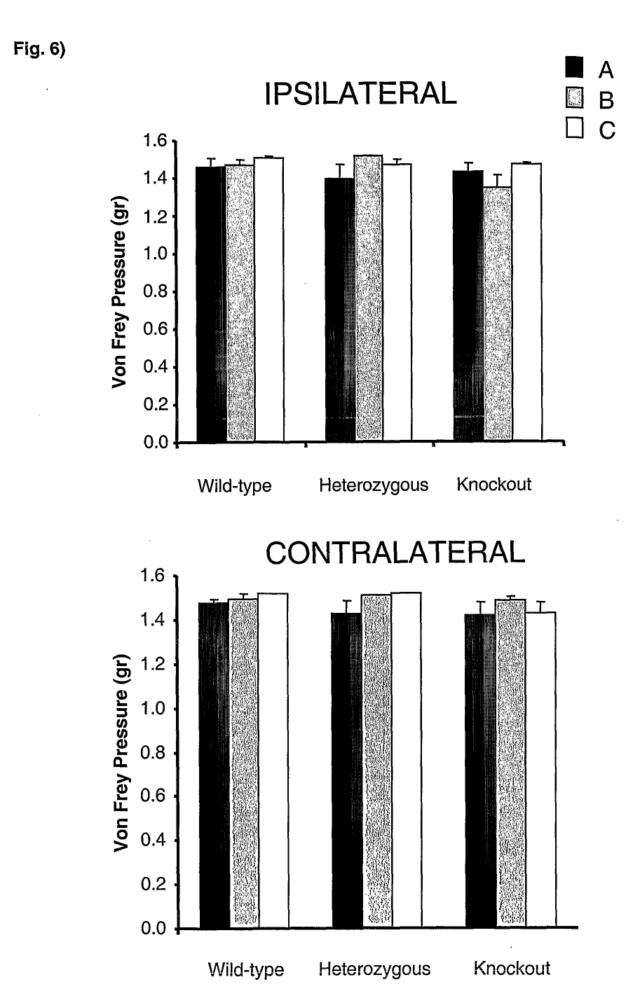

1-21. (canceled)

22. A method for the treatment of mechanical allodynia in a patient, the method comprising administering to said patient a compound binding to the sigma receptor or an administration form of said compound selected from the group consisting of a neutral form, a base form, an acid form, a salt form, a solvate form, a polymorph form, a racemic form, a purified stereoisomer form, and a mixed stereoisomer form in any suitable mixing ratio.

23. The method of claim 22, comprising administering said compound or a physiologically acceptable salt form thereof, a purified enantiomer or diastereomer form thereof, or a mixed enantiomer form or a mixed diastereomer form thereof in any suitable mixing ratio.

24. The method of claim 22, wherein said compound or said administration form thereof has an IC₅₀ value of less than about 5000 nM.

25. The method of claim 24, wherein said compound or said administration form thereof has an IC₅₀ value of less than about 1000 nM.

26. The method of claim 25, wherein said compound or said administration form thereof has an IC50 value of less than about 500 nM.

27. The method of claim 26 or said administration form thereof has an IC₅₀ value of less than about 250 nM.

28. The method claim 27, wherein said compound or said administration form thereof has an IC₅₀ value of less than about 100 nM.

29. The method of claim 28, wherein said compound or said administration form thereof has an IC₅₀ value of less than about 50 nM.

30. The method of claim 22, wherein said compound or said administration form thereof is a sigma receptor antagonist.

31. The method of claim 22, wherein said compound or said administration form thereof is a partial sigma receptor antagonist.

32. The method of claim 22, wherein said compound or said administration form thereof is a sigma receptor inverse agonist.

33. The method of claim 22, wherein said sigma receptor is of the sigma-1 receptor subtype.

34. The method of claim 22, wherein said compound or said administration form thereof is selected from the group consisting of:

| | |
|---|---|
| (−)-Cyanopindolol hemifumarate | (−)-SPARTEINE SULFATE |
| | PENTAHYDRATE |
| (+)-HIMBACINE | (2-Dibutylamino-Ethyl)-Carbamic Acid |
| | 2-(4-Benzofuran-2-Ylmethyl- |
| | Piperazin-1-Yl)-Ethyl Ester |
| (4-[1,2,3]Thiadiazol-4-Yl-Benzyl)- | (S)-Methamphetamine HCl |
| Carbamic Acid 1-(3-Methoxy-2-Nitro- | |
| Benzyl)-Piperidin-3-Ylmethyl Ester | |
| [1-(9-Ethyl-9H-Carbazol-3-Ylmethyl)- | [1-(9-Ethyl-9H-Carbazol-3-Ylmethyl)- |
| Pyrrolidin-3-Yl]-Carbamic Acid 1-(3- | Pyrrolidin-3-Yl]-Carbamic Acid 2- |
| Benzyloxy-4-Methoxy-Benzyl)-Piperidin- | (Tert-Butoxycarbonyl-Naphthalen-1- |
| 3-Ylmethyl Ester | Ylmethyl-Amino)-Ethyl Ester |
| [4-(4-Ethyl-3,5-Dimethyl-Pyrazol-1-Yl)- | 1-(1,2-Diphenylethyl)Piperidine |
| Phenyl]-[4-(3-Phenyl-Allyl)-Piperazin-1- | Maleate, (+/−) |
| Yl]-Methanone | |
| 1-(1-Naphthyl)Piperazine HCl | 1-(3-Chlorophenyl)Piperazine HCl |
| 1-(4-Bromo-Benzenesulfonyl)-4-(2-Tert- | 2-(2-{[1-(3-Chloro-Benzyl)-Pyrrolidin- |
| Butylsulfanyl-Benzyl)-Piperazine | 3-Yl]-Methyl-Carbamoyl}-2-Methyl- |
| | Propyl)-4,6-Dimethyl-Benzoic Acid |
| 2-Chloro-11-(4- | 3,3′-Diethylthiacarbocyanine Iodide |
| Methylpiperazino)Dibenz[B,F]Oxepin Maleate | |
| 3-Mercapto-2-Methylpropanoic Acid | 3-Quinuclidinyl Benzilate |
| 1,2-Diphenylethylamine Salt | |
| 3-Tropanyl-3,5-Dichlorobenzoate | 3-Tropanyl-indole-3-Carboxylate HCl |
| 4-(1H-indol-4-Yl)-Piperazine-1- | 4-(2-Tert-Butylsulfanyl-Benzyl)- |
| Carboxylic Acid 2-(5-Bromo-2-Ethoxy- | Piperazine-1-Carboxylic Acid 2- |
| Phenylamino)-Cyclohexylmethyl Ester | Thiophen-2-Yl-Ethyl Ester |
| 4-(3,5-Dimethoxy-Phenyl)-Piperazine-1- | 4-(3-Nitro-5-Sulfamoyl-Thiophen-2- |
| Carboxylic Acid 1-(2-Fluoro-Benzyl)- | Yl)-Piperazine-1-Carboxylic Acid 1-(2- |
| Piperidin-2-Ylmethyl Ester | Fluoro-5-Methoxy-Benzyl)-Piperidin-3- |
| | Ylmethyl Ester |
| 4-(4-Fluorobenzoyl)-1-(4- | 4-(5-Trifluoromethyl-Pyridin-2-Yl)- |
| Phenylbutyl)Piperidine Oxalate | Piperazine-1-Carboxylic Acid Pent-2- |
| | Ynyl Ester |
| 4,4′-Bis[4-(P-Chlorophenyl)-4- | 4-[1-(4-Chlorobenzyl)-4- |
| Hydroxypiperidino]Butyrophenone | (benzylpiperidin-4-yl]-2-hydroxy-4- |
| | oxobut-2-enoic acid |
| 4-Bromo-N-[1-(9-Ethyl-9H-Carbazol-3- | 4′-Chloro-3-Alpha- |
| Ylmethyl)-Pyrrolidin-3-Yl]-2- | (Diphenylmethoxy)Tropane HCl |
| Trifluoromethoxy-Benzenesulfonamide | |
| 4-Furan-2-Ylmethyl-Piperazine-1- | 4-Methoxy-N-[1-(7-Methoxy- |
| Carboxylic Acid 2-{4-[3-(2- | Benzo[1,3]Dioxol-5-Ylmethyl)- |
| Trifluoromethyl-Phenothiazin-10-Yl)- | Pyrrolidin-3-Yl]-Benzenesulfonamide |
| Propyl]-Piperazin-1-Yl}-Ethyl Ester | |
| 5-(N-Ethyl-N-Isopropyl)-Amiloride | 7-Hydroxy-DPAT HBr, (±)- |
| 8-Hydroxy-DPAT HBr, (R)-(+)- | 8-Hydroxy-DPAT HBr, S(−)- |
| 9-[4-({[4′-(trifluoromethyl-1,1′-biphenyl- | Acepromazine Maleate |
| 2-yl]carbonyl}amino)piperidin-1-yl]-N- | |
| (2,2,2-trifluoroethyl)-9H-fluorene-9- | |
| carboxamide | |
| Acetophenazine Maleate | Acrinol |
| Almaline | Alaproclate HCl |
| Aloe-Emodin | Alprenolol D-Tartrate Salt Hydrate |
| Alprenolol HCl | AMI-193 |
| Aminobenztropine | Amiodarone HCl |
| Amodiaquine HCl | Amorolfine HCl |
| Amoxapine | Anileridine HCl |
| Anisotropine Methylbromide | Anpirtoline |
| ARC 239 DHCl | Asternizole |
| Auramine O HCl | Azaperone |
| Azatadine Maleate | Azelastine HCl |
| Bamethan sulfate | BD 1008 DiHBr |
| BD-1047 | BD-1063 |
| Benextramine TetraHCl | Benfluorex HCl |
| Benidipine HCl | Benoxathian HCl |
| Benoxinate HCl | Benperidol |
| Benproperine Phosphate | Benzododecinium bromide |
| Benzphetamine HCl | Benztropine Mesylate |
| Benzydamine HCl | Bephenium Hydroxynaphthoate |
| Bepridil HCl | Berberine chloride |
| Betaxolol HCl | Bifemelane |
| BMY 7378 DiHCl | Bopindolol Malonate |
| BP 554 Maleate | Bromhexine HCl |
| Bromodiphenhydramine HCl | Bromperidol |
| Brompheniramine Maleate | BTCP HCl |
| Buclizine HCl | Buflomedil HCl |
| Bupropion HCl | Buspirone HCl |
| Butacaine Sulfate | Butaclamol HCl, (±)- |
| Butenafine HCl | Butoconazole Nitrate |
| BW 723C86 HCl | Carbetapentane Citrate |
| Carbinoxamine Maleate | Carplpramine DiHCl DiH2O |
| Carvedilol | Cephapirin Benzathine |
| CGS-12066A Maleate | Chloroprocaine HCl |
| Chloroquine Phosphate | Chlorpheniramine Maleate |
| Chlorphenoxamine HCl | Chlorpromazine HCl |
| Chlorprothixene | Cinanserin HCl |
| Cinnarizine | Cirazoline HCl |
| Cis-(+/−)-N-Methyl-N-[2-(3,4- | Cis(Z)-Flupentixol DiHCl |
| Dichlorophenyl)Ethyl]-2-(1- | |
| Pyrrolidinyl)Cyclohexamine DiHBr | |
| Cisapride Hydrate | Citalopram HBr |
| Clebopride Maleate Salt | Clemastine Fumarate |
| Clemizole HCl | Clenbuterol HCl |
| Clidinium Bromide | Clobenpropit 2HBr |
| Clofazimine | Clofilium Tosylate |
| Clomiphene Citrate | Clomiphene Related Compound A |
| Clomipramine | Cloperastine HCl |
| Clorgyline HCl | Clozapine |
| CONESSINE | Cyclizine |
| Cyclobenzaprine HCl | Cycloheximide |
| Cyproheptadine HCl | Darrow Red HCl |
| Demecarium Bromide | Denatonium Benzoate |
| Deptropine Citrate | Desloratadine |
| Dexbrompheniramine Maleate | Dexchlorpheniramine Maleate |
| Dexfenfluramine HCl | Dibucaine HCl |
| Dicyclomine HCl | Diethylpropion HCl |
| Dimethisoquin HCl | Dimetindene Maleate |
| Diphemanil Methylsulfate | Diphenidol HCl |
| Diphenoxylate HCl | Diphenylpyraline HCl |
| Dipropyldopamine HBr | Dobutamine HCl |
| Donepezil HCl | Doxepin HCl |
| Droperidol | Duloxetine |
| Dyclonine HCl | Ebastine |
| Econazole Nitrate | Epinastine HCl |
| Ethaverine HCl | Ethopropazine HCl |
| Eticlopride HCl, S(−)- | Etofenamate |
| Etonitazenyl Isothiocyanate | Femoxetine HCl |
| Fenfluramine HCl | Fentanyl Citrate |
| Fenticonazole Nitrate | Fipexide HCl |
| Flavoxate HCl | Flunarizine diHCl |
| Fluoxetine Related Compound B | Flupertapine |
| Fluphenazine Decanoate DiHCl | Fluphenazine Enanthate DiHCl |
| Fluphenazine HCl | Fluphenazine N-Mustard DiHCl |
| Flurazepam Related Compound C | Fluspirilene |
| Fluvoxamine Maleate | GBR 12783 DiHCl |
| GBR 12909 DiHCl | GBR 13069 DiHCl |
| GBR-12935 DiHCl | GR 89696 Fumarate |
| Guanabenz Acetate | Guanadrel Sulfate |
| Guanethidine Sulfate | Halofantrine HCl |
| Haloperidol | HEAT HCl |
| Hexylcaine HCl | Hycanthone |
| Hydroxychloroquine Sulfate | Hydroxyzine HCl |
| Hyoscyamine Sulfate | IBZM, S(−)- |
| ICI-199,441 HCl | Ifenprodil Tartrate |
| Imlpramine HCl | Indatraline HCl |
| Iofetamine HCl | Irinotecan HCl |
| Isamoltane Hemifumarate | Isopromethazine HCl |
| Isoxsuprine HCl | Ketanserin L-Tartrate |
| Ketoconazole | Ketotifen Fumarate Salt |
| L-693,403 Maleate | L-741,626 |
| L-741,742 HCl | L-745,870 TriHCl |
| Labetalol HCl | Levetimide HCl, R(−) |
| Levobunolol HCl | Lidoflazine |
| Lisuride Hydrogen Maleate, R(+)- | Lobeline HCl |
| Iomerizine diHCl | Loperamide HCl |
| Loxapine Succinate | LY-53,857 Maleate |
| Maprotiline HCl | Mazindol |
| MDL 12,330A HCl | Mebhydroline 1,5- |
| | naphthalendisulfonate Salt |
| Meclizine HCl | Mefloquine HCl |
| Meprylcaine HCl | Mesoridazine Besylate |
| Metaphit Methanesulfonate | Metergoline |
| Methantheline Bromide | Methdilazine |
| Methiothepin Mesylate | Methlxene HCl |
| Methoctramine | Methotrimeprazine Maleate |
| Methylene Violet 3Rax HCl | Metipranolol |
| Mexiletine HCl | Mianserin HCl |
| Miconazole | ML-9 HCl |
| Morantel Hydrogen L-Tartrate | MR 16728 HCl |
| N-(2-Chloroethyl)-N-Ethyl-2- | N′-[2-(Benzo[1,2,5]Thiadiazole-4- |
| Bromobenzylamine HCl | Sulfonylamino)-Acetyl]- |
| | Hydrazinecarboxylic Acid 2-(2-{4-[(4- |
| | Chloro-Phenyl)-Phenyl-Methyl]- |
| | Piperazin-1-Yl}-Ethoxy)-Ethyl Ester |
| Nafronyl Oxalate Salt | Naftifine |
| Naftopidil diHCl | Naltriben Mesylate |
| NAN-190 HBr | NE-100 |
| Nefazodone | Nefopam HCl |
| Nicardipine HCl | Nicergoline |
| Niguldipine HCl, (+/−)- | Nisoxetine HCl |
| Nortriptyline HCl | Nylidrin HCl |
| Octoclothepin Maleate, (±)- | Orphenadrine Citrate |
| Oxamniquine | Oxamniquine Related Compound A |
| Oxamniquine Related Compound B | Oxatomide |
| Oxiconazole Nitrate | Oxybutynin HCl |
| Panaxatriol | PAPP |
| Paroxetine | Paxilline |
| p-Chlorobenzhydrylpiperazine | Penbutolol Sulfate |
| Pentamidine Isethionate | Pentazocine, (±)- |
| Pergolide Methanesulfonate | Perhexiline Maleate Salt |
| Perospirone | Perphenazine |
| Perphenazine Sulfoxide | Phenamil Methanesulfonate |
| Phencyclidine HCl | Phenosafranin HCl |
| Phenoxybenzamine HCl | Phenyltoloxamine Citrate Salt |
| Piboserod | Pimozide |
| Pinacyanol chloride | Pindobind, (+/−)- |
| Piperacetazine | Piperazine-1,4-Dicarboxylic Acid |
| | Benzyl Ester 2-[4-(4-Dimethylamino- |
| | Benzyl)-Piperazin-1-Yl]-Ethyl Ester |
| Piperidolate HCl | Pirenperone |
| PPHT HCl, (±)- | Pramoxine HCl |
| Prenylamine Lactate Salt | Pridinol Methanesulfonate Salt |
| Prochlorperazine Maleate | Procyclidine HCl |
| Proflavine Hemisulfate Salt | Progesterone |
| Promazine HCl | Promethazine HCl |
| Propafenone HCl | Proparacaine HCl |
| Propericyazine | Propiomazine |
| Propranolol HCl | Protokylol |
| Protriptyline HCl | Pyrilamine Maleate |
| Pyrimethamine | Pyrrolidine-1,2-Dicarboxylic Acid 1-[1- |
| | (4-Allyloxy-Benzyl)-Piperidin-2- |
| | Ylmethyl] Ester 2-Benzyl Ester |
| Pyrvinium Pamoate | Queliapine Fumarate |
| Quinacrine HCl | Quinaidine Red |
| Quipazine Dimaleate | Quipazine, 6-Nitro-, Maleate |
| Raloxifene | Rimantadine HCl |
| Risperidone | Ritanserin |
| Ritodrine HCl | RS 23597-190 HCl |
| RS 67333 HCl | RS 67506 HCl |
| Safranin O HCl | Salmeterol |
| SB203186 | SCH-23390 HCl, R(+)- |
| Sertaconazole Nitrate | Sertindole |
| Sertraline | Sibutramine HCl |
| SKF-525A HCl | SKF-96365 HCl |
| SNC 121 | Spiperone HCl |
| Sufentanil | T-226296 |
| Tamoxifen Citrate | Tamsulosin HCl |
| Tegaserod Maleate | Terbinafine HCl |
| Terconazole | Terlenadine |
| Terfenadine Related Compound A | Tetracaine HCl |
| Tetrindole Mesylate | Thlethylperazine Malate |
| Thioperamide Maleate | Thioproperazine |
| Thioridazine | Thiothixene |
| Thiothixene, (E)- | Thonzonium Bromide |
| Tloconazole Related Compound A | TMB-8 HCl |
| Tolterodine L-Tartrate | Toremifene Citrate |
| Tramazoline HCl | Trans-U-50488 Methanesulfonate, |
| | (±)- |
| Trazodone HCl | Tridihexethyl Chloride |
| Trifluoperazine HCl | Trifluperidol HCl |
| Triflupromazine HCl | Trihexyphenldyl HCl |
| Trimebutine | Trimeprazine Hemi-L-Tartrate |
| Trimipramine Maleate | Tripelennamine HCl |
| Triprolidine HCl | Triprolidine HCl Z Isomer |
| Tropanyl 3,5-Dimethylbenzoate | Tropine 2-(4- |
| | Chlorophenoxy)Butanoate, Maleate |
| U-50488 HCl, (−)- | U-62066 |
| UH 232 Maleate, (+)- | Vecuronium Bromide |
| Verapamil HCl | Verapamil Related Compound B |
| Vesamicol HCl | Vinpocetine |
| W-7 HCl | WB-4101 HCl |
| Xylazine | Xylometazoline HCl |

35. The method of claim 22, wherein said compound has formula IB 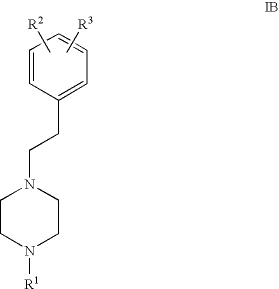
or an administration form thereof selected from the group consisting of a neutral form, a base form, an acid form, a salt form, a solvate form, a polymorph form, a racemic form, a purified stereoisomer form, and a mixed stereoisomer form in any suitable mixing ratio,
wherein
R¹ is optionally substituted, optionally branched, and optionally unsaturated C₁₋₆ alkyl; and
R² and R³ are independently selected from the group consisting of —H; —OH; —SH; —NH₂; optionally substituted, optionally branched, and optionally unsaturated C₁₋₆ alkyl; halo; and optionally substituted, optionally branched, and optionally unsaturated —O—C₁₋₆ alkyl.

36. The method of claim 35, wherein R¹ is optionally branched and optionally unsaturated C₁₋₆ alkyl, which is optionally substituted with one or more substituents independently selected from the group consisting of —F, —Cl, —Br, —I, —NH₂, —SH, and —OH.

37. The method of claim 35, wherein R² and R³ are independently selected from the group consisting of —H; optionally branched and optionally unsaturated C₁₋₆ alkyl, which is optionally substituted with one or more substituents independently selected from the group consisting of —F, —Cl, —Br, —I, —NH₂, —SH, and —OH; halo; and optionally branched and optionally unsaturated —O—C₁₋₆ alkyl, which is optionally substituted with one or more substituents independently selected from the group consisting of —F, —Cl, —Br, —I, —NH₂, —SH, and —OH.

38. The method of claim 35, wherein R¹ is optionally substituted, optionally branched, and optionally unsaturated C₁₄ alkyl.

39. The method of claim 38, wherein R¹ is optionally substituted, optionally branched, and saturated C₁₋₄ alkyl.

40. The method of claim 39, wherein R¹ is unsubstituted, optionally branched, and saturated C₁₋₄ alkyl.

41. The method of claim 40, wherein R¹ is selected from the group consisting of —CH₃, —C₂H₅, —C₃H₇, and —C₄H₉.

42. The method of claim 35, wherein R² and R³ are independently selected from the group consisting of —H; —OH; —SH; —NH₂; optionally substituted, optionally branched, and optionally unsaturated C₁₋₄ alkyl; halo; and optionally substituted, optionally branched, and optionally unsaturated —O—C₁₋₄ alkyl.

43. The method of claim 42, wherein R² and R³ are independently selected from. the group consisting of —H; —OH; —NH₂; optionally substituted, optionally branched, and saturated C₁₋₄ alkyl; —F; —Cl; —Br; —I; optionally substituted, optionally branched, and saturated —O—C₁₋₄ alkyl.

44. The method of claim 43, wherein R² and R³ are independently selected from the group consisting of —H, —OH, —NH₂, —CH₃, —C₂H₅, —C₃H₇, —C₄H₉, —CF₃, —CHF₂, —F, —Cl, —Br, —I, —O—CH₃, —O—C₂H₅, —O—C₃H₇, and —O—C₄H₉.

45. The method of claim 44, wherein R² and R³ are independently selected from the group consisting of —H, —F, —Cl and —CF₃.

46. The method of claim 35, wherein R² and R³ are attached at the 3- and 4-positions of the phenyl ring, respectively.

47. The method of claim 22, wherein said compound is 1-(3,4-dichlorophenethyl)-4-methylpiperazine.

48. The method of claim 22, wherein said compound is N1-(3,4-dichlorophenethyl)-N1,N2,N2-trimethylethane-1,2-diamine.
